(19) United States
(12) Patent Application Publication
(10) Pub. No.: US 2007/0189547 A1
(43) Pub. Date: Aug. 16, 2007

(54) METHOD AND APPARATUS FOR ECHO CANCELLATION

(75) Inventors: Wei-hao Hsu, Kaohsiung City (TW); Hsi-Wen Nien, Hsinchu County (TW)

Correspondence Address: THOMAS, KAYDEN, HORSTEMEYER & RISLEY, LLP, 100 GALLERIA PARKWAY, NW, STE 1750, ATLANTA, GA, 30339-5948, US

(73) Assignee: MEDIATEK INC., Hsin-Chu (TW)

(21) Appl. No.: 11/624,710

(22) Filed: Jan. 19, 2007

Related U.S. Application Data
(60) Provisional application No. 60/762,704, filed on Jan. 27, 2006.

(51) Int. Cl. H04M 9/08; A61F 11/06; G10K 11/16; H03B 29/00

(52) U.S. Cl. 381/71.1; 379/406.01

(57) ABSTRACT

Method and apparatus for echo cancellation are provided. In an echo cancellation device, remote and local signals are separated by frequency to generate a plurality of remote and local sub-band signals each corresponding to a sub-band. A plurality of voice activity detectors each respectively receives remote and a local sub-band signals to detect voice activity of the corresponding sub-band. A plurality of filters each learns a corresponding remote sub-band signal to filter a corresponding local sub-band signal, and generates a filter output of the corresponding sub-band. The learning of remote sub-band signal is dependent on a detection result of the corresponding voice activity detector. A synthesizer is coupled to the plurality of filters, mixing the filter outputs therefrom to generate an echo cancellation result.

### CROSS REFERENCE TO RELATED APPLICATIONS

This application claims the benefit of U.S. Provisional Application No. 60/762,704, filed Jan. 27, 2006.

### BACKGROUND OF THE INVENTION

1. Field of the Invention

The invention relates to echo cancellation, and in particular, to sub-band echo cancellation with voice activity detection.

2. Description of the Related Art

FIG. 1 shows a conventional voice interaction device comprising both a speaker 102 and a microphone 104, such as a telephone. A remote signal x(n) is amplified by the speaker 102 to generate an audible output #OUT. Local input #IN is received by microphone 104 and sent to remote. The microphone 104, however, also receives unwanted background noise #ENV and audible output #OUT along with the local input #IN to generate a mixed result local signal #MIX. Echo effect is induced by the audible output #OUT, reducing communication quality, and an echo canceller 150 is provided to cancel the echo based on a coefficient learned from the remote signal x(n). In the echo canceller 150, a first band separator 106 and a second band separator 108 individually separate the remote signal x(n) and local signal #MIX by frequencies, thus remote sub-band voices R₁ to R₄, and local sub-band voices L₁ to L₄ are respectively generated, each corresponding to a sub-band. The synthesizer 120 then mixes the filter outputs e₁ to e₄ output from the filters 110, to generate an echo cancellation result e(n).

Generally, voice transmission is subsequently distributed around 500 to 1500 Hz, and the local input #IN or audible output #OUT may comprise major distribution only at a specific sub-band. Since most of the sub-bands are less significant noises, separately filtering each sub-band is more efficient than filtering the total band at once. Additionally, the background noise #ENV may also affect filter performance, decreasing coefficient convergence rate. Thus estimation of background noise #ENV is critical. The filters 110 may adaptively utilize various step sizes for different conditions such as double talk, remote talk and local talk. A mechanism to correctly distinguish the conditions is also desirable.

### BRIEF SUMMARY OF THE INVENTION

A detailed description is given in the following embodiments with reference to the accompanying drawings.

An exemplary embodiment of an echo cancellation device is provided, for use in a voice interaction device simultaneously outputting a remote signal while receiving a local signal. The local signal comprises an echo generated from the remote signal. In the echo cancellation device, a first band separator separates the remote signal by frequency to generate a plurality of remote sub-band signals, each corresponding to a sub-band. A second band separator separates the local signal by frequency to generate the same plurality of local sub-band signals, each corresponding to a sub-band. A plurality of voice activity detectors each coupled to a first band separator and a second band separator, respectively receives remote and a local sub-band signals to detect voice activity of the corresponding sub-band. A plurality of filters are individually coupled to a corresponding voice activity detector, learning a corresponding remote sub-band signal to filter a corresponding local sub-band signal, and generating a filter output of the corresponding sub-band. The learning of remote sub-band signal is dependent on a detection result of the corresponding voice activity detector. A synthesizer is coupled to the plurality of filters, mixing the filter outputs therefrom to generate an echo cancellation result.

The echo cancellation device may further comprise a controller, detecting double talk to generate a double talk flag base on the remote signal and the local signal. Voice activity detectors are coupled to the controller, each generating an activation flag based on the double talk flag, and voice activities of first and local sub-band signals. Each of the filters comprises a coefficient set recursively updated by normalized least mean square (NLMS) algorithm. If the activation flag is a first value, the filters stop updating the coefficient set.

In each voice activity detector, a remote activity detector detects voice activity of a remote sub-band signal to generate a remote activity flag. A local activity detector detects voice activity of a local sub-band signal to generate a local activity flag. A decision unit receives the remote activity flag, the local activity flag and the double talk flag to generate the activation flag accordingly. If the double talk flag indicates double talk positive, the activation flag is set to the first value. If the double talk flag indicates no double talk, and the remote activity flag and local activity flag indicate that both remote sub-band signal and local sub-band signals are active, the activation flag is set to the first value.

The remote activity detector may estimate a remote or local background noise level, and voice activity of a remote or local sub-band signal is detected if energy level thereof exceeds a certain ratio of the remote or local background noise level.

The echo cancellation device may further comprise a plurality of comfort noise generators, each coupled to a filter, receiving and amplifying a corresponding filter output by control of the controller, and adding comfort noise to the filter output before output to the synthesizer. The echo cancellation device may further comprise an attenuator coupled to the controller, controlled by the controller to determine whether to convert the remote signal to audible output. The controller detects voice activity of the remote signal. If the remote signal is deemed inactive, the controller activates the attenuator to prevent remote signal output, such that the audible output is not generated.

### BRIEF DESCRIPTION OF THE DRAWINGS

The invention can be more fully understood by reading the subsequent detailed description and examples with references made to the accompanying drawings, wherein:

FIG. 1 shows a conventional voice interaction device;

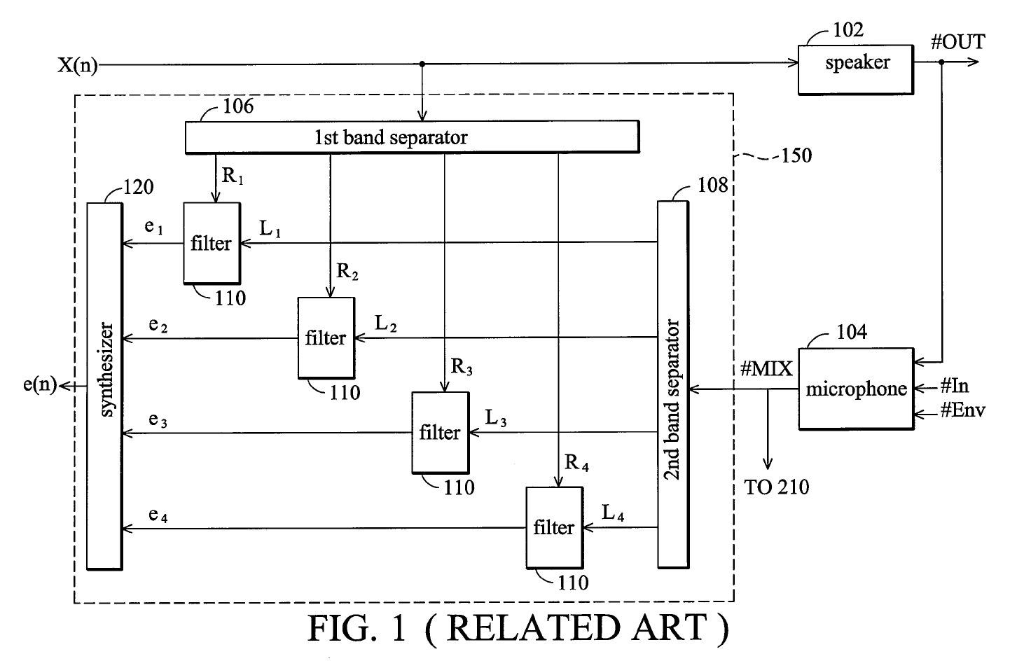

FIG. 2 shows an embodiment of a voice interaction device;

FIG. 3 shows an embodiment of a voice activity detector 300 according to FIG. 2;

FIG. 4 is a flowchart of echo cancellation with voice activity detection; and

FIG. 5 is a flowchart of voice activity detection with background noise level estimation.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

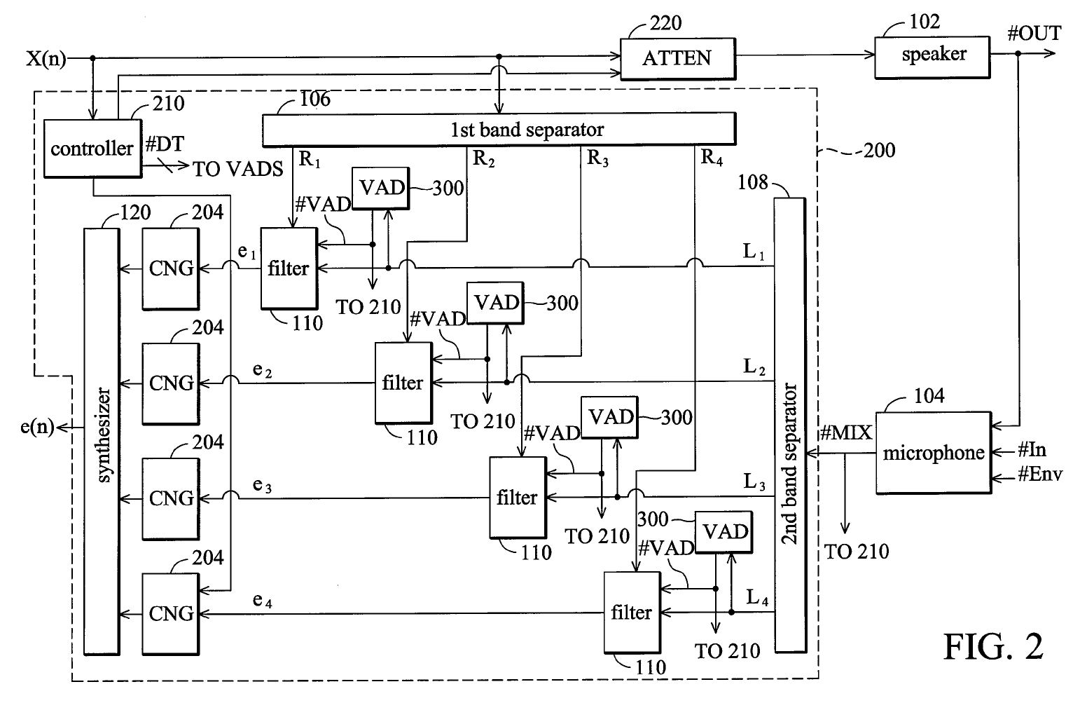

FIG. 2 shows an embodiment of a voice interaction device utilizing echo canceller 200. The frequency response of remote signal x(n) may vary with time, thus the audible output #OUT fed back also changes. The significant vocal frequency may only be distributed at a narrow frequency band, thus at most one or two filters 110 may require high filter performance while others remain inactive. In the embodiment, a plurality of voice activity detectors 300 are added to each sub-band, detecting voice activities of corresponding remote and local sub-band signals Rᵢ and Lᵢ (i ranges from 1 to 4). As an example, the total frequency ranges from 0 to 4 KHz, and four filters 110 are provided for sub-bands of 0 to 1 KHZ, 1 to 2 KHz, 2 to 3 KHz and 3 to 4 KHz. Each filter 110 recursively updates a coefficient set, and the voice activity detectors 300 determine whether to proceed or stop the updates. Specifically, when double talk is detected, the coefficient sets stop updating. For each sub-band, the filters 110 update their coefficient set only when both remote and local activities are positive while double talk is negative. In this way, the total echo cancellation performance can be enhanced, reducing error rate. The filters 110 generate filter outputs eᵢ, thereafter mixed in the synthesizer 120 to generate the echo cancellation result e(n).

In the embodiment, a controller 210 is provided to dominate the voice activity detection. The controller 210 detects double talk by the local signal #MIX and the remote signal x(n) in a conventional fashion, and a double talk flag #DT is generated thereby to indicate the detection result. The voice activity detectors 300 individually receive the double talk flag #DT, and further generate activation flags #VAD to control coefficient update of filters 110 by comparing the double talk flag #DT, and the voice activity of remote and local sub-band signals Rᵢ and Lᵢ. If the activation flag #VAD is a first value, the filters 110 stop updating the coefficient set. Additionally, the filter outputs e₁ to e₄ are individually sent to four comfort noise generators 204 before mixing by the synthesizer 120. The comfort noise generators 204 amplify each filter output eᵢ by control of the controller 210, and add comfort noise to the filter output eᵢ before output to the synthesizer 120. The comfort noise generator 204 can utilize conventional parts.

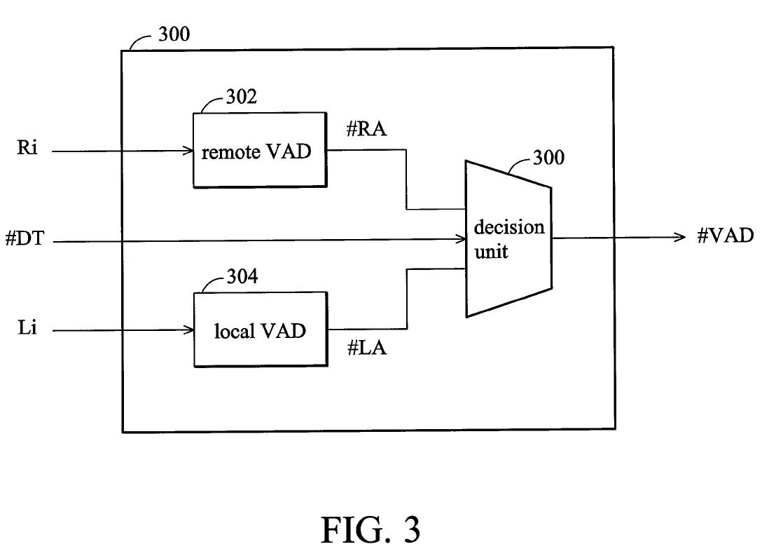

FIG. 3 shows an embodiment of a voice activity detector 300 according to FIG. Each of the voice activity detectors 300 comprises a remote activity detector 302, a local activity detector 304 and a decision unit 306. The remote activity detector 302 receives a remote sub-band signal Rᵢ, detecting voice activity thereof to generate a remote activity flag #RA. The local activity detector 304 receives a local sub-band signal Lᵢ, detecting voice activity thereof to generate a local activity flag #LA. The decision unit 306 compares the remote activity flag #RA, local activity flag #LA and the double talk flag #DT to generate the activation flag #VAD accordingly. The rule is, if the double talk flag #DT indicates double talk positive, the activation flag #VAD is set to the first value. Alternatively, if the double talk flag #DT indicates no double talk, and the remote activity flag #RA and local activity flag #LA indicate that both remote and local sub-band signals Lᵢ and Rᵢ are active, the activation flag #VAD is also set to the first value. The filters 110 stop updating the coefficient set when the activation flag #VAD is the first value. This may imply that a NLMS step size for updating the coefficient set is set to zero. In this way, the filters 110 continuously filter the local sub-band signals Lᵢ irrespective of whether the remote sub-band signal Rᵢ is being learned or not. The remote activity detector 302 estimates a remote background noise level, whereas the local activity detector 304 estimates a local background noise level. Voice activities of remote and local sub-band signals Rᵢ and Lᵢ are detected if energy levels thereof exceed certain ratios of the corresponding background noise levels.

As an example, a running average algorithm is used to estimate the local and remote background noise levels. Remote background noise level is expressed as:

E_{br}(n)=εᵣ·E_{Ri}(n)+(1−εᵣ)·E_{br}(n−1)

where E_{br}(n) is the current remote background noise level, E_{br}(n−1) is previous remote background noise level, εᵣ is a predetermined weighting factor for the remote sub-band signal Rᵢ, and E_{Ri}(n) is the energy of current remote sub-band signal Rᵢ. The weighting factor εᵣ is increased when double talk flag #DT indicates no double talk, or reduced when double talk flag #DT indicates double talk positive. The voice activity is detected as follows:

εE_{Ri}(n)>α·E_{br}(n), V_{Ri}=1

εE_{Ri}(n)≦α·E_{br}(n), V_{Ri}=0

where α is a programmable threshold level, and the V_{Ri} means voice activity of remote sub-band signal Rᵢ, 0 as negative, and 1 as positive. Similarly for local background noise level:

E_{bl}(n)=εₗ*E_{Li}(n)+(1−εₗ)·E_{bl}(n−1)

where E_{bl}(n) is the current local background noise level, E_{bl}(n−1) is previous local background noise level, εₗ is a predetermined weighting factor for the Lᵢ, and E_{Li}(n) is the energy of current Lᵢ. The weighting factor εₗ is increased when double talk flag #DT indicates no double talk, and reduced when double talk flag #DT indicates double talk positive. The voice activity is detected as follows:

εE_{Li}(n)>β·E_{bl}(n), V_{Li}=1

εE_{Li}(n)≦β·E_{bl}(n), V_{Li}=0

where β is a programmable threshold level, and the V_{Li} means voice activity of Li, 0 as negative, and 1 as positive.

The remote activity flag #RA output from remote activity detector 302 may further be fed back to the controller 210. In FIG. 2, an attenuator 220 is coupled to the speaker 102, and controlled by the controller 210 to determine whether to pass the remote signal x(n) to the speaker 102. If all the remote activity flag #RA are negative, the attenuator 220 blocks the remote signal x(n) from being sent to speaker 102, thus the audible output #OUT is not generated. Alternatively, the voice activity of remote signal x(n) can be directly detected in the controller 210.

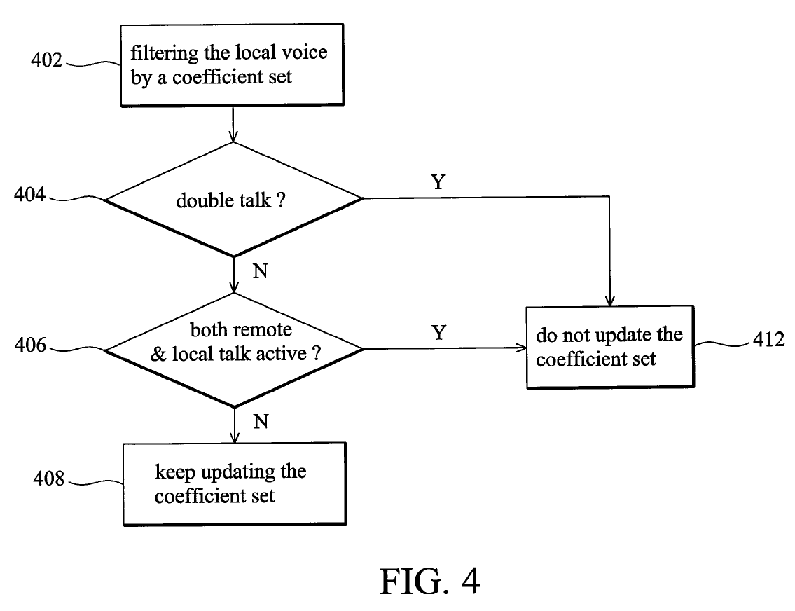

FIG. 4 is a flowchart of echo cancellation with voice activity detection. In step 402, the echo canceller 200 continuously processes echo cancellation from the remote signal x(n) and local signal #MIX. In step 404, it is determined whether double talk is present. If so, step 412 is processed, and coefficients of all the filters 110 are not updated while generating the filter outputs eᵢ. In step 406, voice activities of remote sub-band signal Rᵢ and local sub-band signals Lᵢ are individually examined. In step 412, for a filters 110, if both remote and local sub-band signals Rᵢ and Lᵢ are active, it is deemed a pure echo condition, and the coefficient set therein is not updated. Otherwise, the filters 110 keep updating the coefficient sets in step 408.

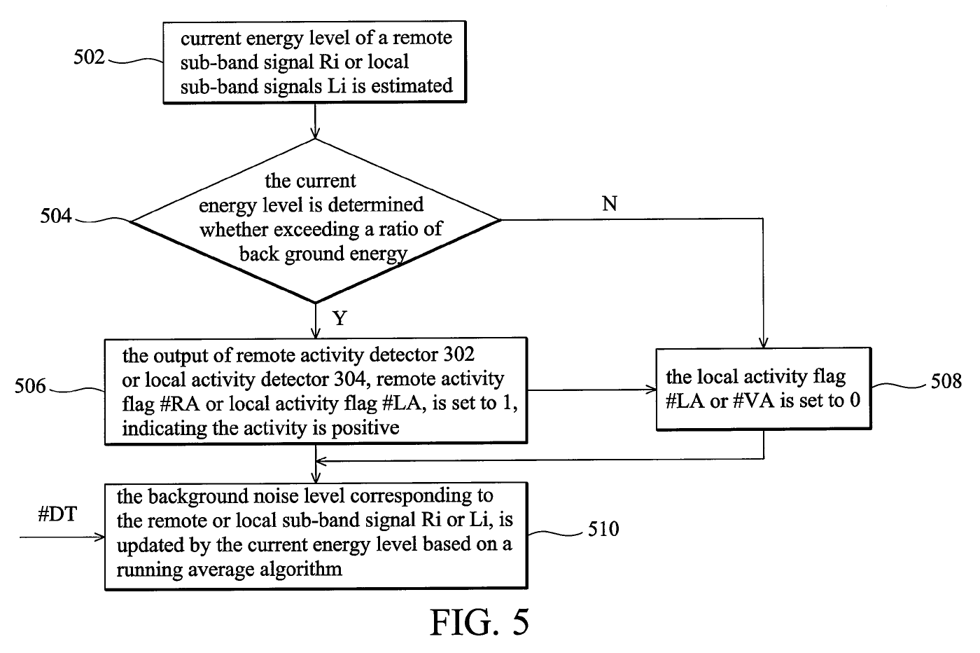

FIG. 5 is a flowchart of voice activity detection with background noise level estimation. In step 502, current energy level of a remote sub-band signal Rᵢ or local sub-band signals Lᵢ is estimated. In step 504, it is determined whether the current energy level exceeds a ratio of background energy. If so, in step 506, the output of remote activity detector 302 or local activity detector 304, remote activity flag #RA or local activity flag #LA, is set to 1, indicating the activity is positive. If not, in step 508, the local activity flag #LA or #VA is set to 0. In step 510, the background noise level corresponding to the remote or local sub-band signal Rᵢ or Lᵢ is updated by the current energy level based on a running average algorithm. The weighting factor of the running average level is dependent on the double talk flag #DT sent from the controller 210.

The embodiment can be an applied for a mobile phone, or any devices simultaneously comprising a microphone and a speaker. The blocks illustrated in FIG. 2 and FIG. 3 can be logic units implemented by circuit or software programs. The echo canceller 200 can also be algorithm implemented by a DSP cooperating with memory devices. As an example, if the embodiment is a VOIP application, the echo canceller 200 can be a software module installed in an embedded system such as Linux.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An echo cancellation device for a voice interaction device simultaneously outputting a remote signal while receiving a local signal, wherein the local signal comprises an echo generated from the remote signal, the echo cancellation circuit comprising:
a first band separator, separating the remote signal by frequency to generate a plurality of remote sub-band signals each corresponding to a sub-band;
a second band separator, separating the local signal by frequency to generate the same plurality of local sub-band signals each corresponding to a sub-band;
a plurality of voice activity detectors each coupled to a first band separator and a second band separator, respectively receiving a remote sub-band signal and a local sub-band signal to detect voice activity of the corresponding sub-band;
a plurality of filters each coupled to a voice activity detector, learning a corresponding remote sub-band signal to filter a corresponding local sub-band signal, and generating a filter output of the corresponding sub-band; wherein the learning of remote sub-band signal is dependent on a detection result of the corresponding voice activity detector; and
a synthesizer, coupled to the plurality of filters, mixing the filter outputs therefrom to generate an echo cancellation result.

2. The echo cancellation device as claimed in claim 1, further comprising:
a controller, detecting double talk to generate a double talk flag base on the remote signal and the local signal; wherein:
the voice activity detectors are coupled to the controller, each generating an activation flag based on the double talk flag, voice activities of first and local sub-band signals;
each of the filters comprises a coefficient set recursively updated by normalized least mean square (NLMS) algorithm; and
if the activation flag is a first value, the filters stop updating the coefficient set.

3. The echo cancellation device as claimed in claim 2, wherein each voice activity detector comprises:
a remote activity detector, detecting voice activity of a remote sub-band signal to generate a remote activity flag;
a local activity detector, detecting voice activity of a local sub-band signal to generate a local activity flag;
a decision unit, receiving the remote activity flag, the local activity flag and the double talk flag to generate the activation flag accordingly; wherein:
if the double talk flag indicates double talk positive, the activation flag is set to the first value; and
if the double talk flag indicates no double talk, and the remote activity flag and local activity flag indicate that both remote sub-band signal and local sub-band signals are active, the activation flag is set to the first value.

4. The echo cancellation device as claimed in claim 3, wherein:
the remote activity detector estimates a remote background noise level; and
voice activity of a remote sub-band signal is detected if energy level thereof exceeds a first ratio of the remote background noise level.

5. The echo cancellation device as claimed in claim 4, wherein the remote background noise level is updated by a running average algorithm as:
E_{b}(n)=ε·E_{Ri}(n)+(1−ε)·E_{b}(n−1)
where E_{b}(n) is the current remote background noise level, E_{b}(n−1) is previous remote background noise level, ε is a weighting factor, and E_{Ri}(n) is the current energy of an i^{th} remote sub-band signal;
the remote activity detector increases the weighting factor when the double talk flag indicates no double talk; and
the remote activity detector reduces the weighting factor when the double talk flag indicates double talk positive.

6. The echo cancellation device as claimed in claim 3, wherein:
the local activity detector estimates a local background noise level; and
voice activity of a local sub-band signal is detected if energy level thereof exceeds a second ratio of the local background noise level.

7. The echo cancellation device as claimed in claim 6, wherein the local background noise level is updated by a running average algorithm as:
E_{b}(n)=ε*E_{Li}(n)+(1−ε)·E_{b}(n−1)
where E_{b}(n) is the current local background noise level, E_{b}(n−1) is previous local background noise level, ε is a weighting factor, and E_{Li}(n) is the current energy of a i^{th} local sub-band signal;
the local activity detector increases the weighting factor when the double talk flag indicates no double talk; and
the local activity detector reduces the weighting factor when the double talk flag indicates double talk positive.

8. The echo cancellation device as claimed in claim 2, further comprising a plurality of comfort noise generators, each coupled to a filter, receiving and amplifying a corresponding filter output by control of the controller, and adding comfort noise to the filter output before output to the synthesizer.

9. The echo cancellation device as claimed in claim 2, further comprising an attenuator, coupled to the controller, controlled by the controller to determine whether to convert the remote signal to an audible output, wherein:
the controller detects voice activity of the remote signal; and
if the remote signal is deemed inactive, the controller activates the attenuator to stop the remote signal output, such that the audible output is not generated.

10. An echo cancellation method for a voice interaction device simultaneously outputting a remote signal while receiving a local signal, wherein the local signal comprises an echo generated from the remote signal, the echo cancellation method comprising:
filtering the remote signal by frequency to generate a plurality of remote sub-band signals each corresponding to a sub-band;
filtering the local signal by frequency to generate the same plurality of local sub-band signals each corresponding to a sub-band;
detecting voice activities of a remote sub-band signal and a local sub-band signals corresponding to a sub-band;
learning the remote sub-band signal by NLMS algorithm to generate a coefficient set;
filter the local sub-band signals by the coefficient set to generate a filter output; wherein the coefficient set is updated according to the voice activity detection result; and
mixing the filter outputs from all sub-bands to generate an echo cancellation result.

11. The echo cancellation method as claimed in claim 10, further comprising:
detecting double talk base on the remote signal and the local signal to generate a double talk flag;
generating an activation flag based on the double talk flag, the voice activities of remote and local signals; and
if the activation flag is a first value, the filters stop updating the coefficient set.

12. The echo cancellation method as claimed in claim 11, wherein detection of the voice activity comprises:
detecting voice activity of a remote sub-band signal to generate a remote activity flag;
detecting voice activity of a local sub-band signal to generate a local activity flag;
generating the activation flag from the remote activity flag, the local activity flag and the double talk flag; wherein:
if the double talk flag indicates double talk positive, the activation flag is set to the first value;
if the double talk flag indicates no double talk, and the remote and local activity flags indicate that both remote and local sub-band signals are active, the activation flag is set to the first value; and
otherwise the activation flag is set to a second value that disables the coefficient update.

13. The echo cancellation method as claimed in claim 12, wherein detection of the voice activities further comprises:
estimating a remote background noise level; and
if energy level of a remote sub-band signal exceeds a first ratio of the remote background noise level, confirming voice activity of the remote sub-band.

14. The echo cancellation method as claimed in claim 13, wherein:
the remote background noise level is updated by a running average algorithm as:
E_{b}(n)=ε·E_{Ri}(n)+(1−ε)·E_{b}(n−1)
where E_{b}(n) is the current remote background noise level, E_{b}(n−1) is previous remote background noise level, ε is a weighting factor, and E_{Ri}(n) is the current energy of an i^{th} remote sub-band signal;
the estimation of remote background noise level comprises:
increasing the weighting factor when double talk flag indicates no double talk; and
reducing the weighting factor when double talk flag indicates double talk positive.

15. The echo cancellation method as claimed in claim 12, wherein detection of voice activity further comprises:
estimating a local background noise level; and
if energy level of a local sub-band signal exceeds a second ratio of the local background noise level, confirming voice activity of the local sub-band signal.

16. The echo cancellation method as claimed in claim 15, wherein:
the local background noise level is updated by a running average algorithm as:
E_{b}(n)=ε·E_{Li}(n)+(1−ε)·E_{b}(n−1)
where E_{b}(n) is the current local background noise level, E_{b}(n−1) is previous local background noise level, ε is a weighting factor, and E_{Li}(n) is the current energy of an i^{th} local sub-band signal;
the estimation of local background noise level comprises:
increasing the weighting factor when the double talk flag indicates no double talk; and
reducing the weighting factor when the double talk flag indicates double talk positive.

17. The echo cancellation method as claimed in claim 11, further comprising adding comfort noise to the filter outputs before mixing.

18. The echo cancellation method as claimed in claim 11, further comprising:
determining whether to amplify the remote signal to generate an audible output based on voice activity of the remote signal; and
if remote signal is deemed inactive, stopping the remote signal from being converted to the audible output.
